(19) United States
(12) Patent Application Publication
(10) Pub. No.: US 2022/0220087 A1
(43) Pub. Date: Jul. 14, 2022

(54) ANTHOCYANIN EXTRACTION METHODS

(71) Applicant: Francis Michelle Mann, Oak Creek, WI (US)

(72) Inventor: Francis Michelle Mann, Oak Creek, WI (US)

(21) Appl. No.: 17/605,845

(22) PCT Filed: Apr. 23, 2020
(86) PCT No.: PCT/US2020/029541

§ 371 Date: Oct. 22, 2021

Related U.S. Application Data
(60) Provisional application No. 62/837,317, filed on Apr. 23, 2019.

(51) Int. Cl. C07D 311/04; C12P 17/06

(52) U.S. Cl. CPC C07D 311/04; C12P 17/06

(57) ABSTRACT

A method to prepare and/or isolate water soluble anthocyanins is provided.

### CROSS-REFERENCE TO RELATED APPLICATIONS

This application claims the benefit of the filing date of U.S. Application No. 62/837,317, filed on Apr. 23, 2019, the disclosure of which is incorporated by reference herein.

### BACKGROUND

Polyphenolic antioxidants are useful compounds in the food ingredient, supplement, and fine chemical industries. One family of polyphenolic antioxidants are the anthocyanins. Anthocyanins are found in plants, e.g., in fruits and flowers, and they exist in soluble and insoluble forms. Specifically, they exist as glycoconjugates and polymers (proanthocyanidins) within fruits and flowers, while free (soluble) anthocyanins are both more bioavailable and have higher antioxidant capacity. Due to their intense pigment, e.g., pink and purple, anthocyanins also act as natural dyes.

Berries are exceptionally high in anthocyanins, however 30-40% of the anthocyanin content is retained in the fruit solids, e.g., skin, seeds, and pulp upon extraction. For instance, cranberry pulp contains membranes, complex carbohydrates (e.g. pectin, pullulan, and cellulose), proteins, anthocyanin conjugates, and proanthocyanidins (e.g., polymeric anthocyanins). Processing of fruit for commercial food sale results in a considerable amount of fruit waste (e.g., skin, seeds, pulp, and whole fruit deemed unfit for human consumption). For example, in the State of Wisconsin, the cranberry crop is estimated to generate 500 million pounds of cranberries annually and it was estimated that 2 million pounds of cranberry waste was generated at a single facility within a single year. This waste material might serve as a unique source of anthocyanins if the residual anthocyanins could be freed from the fruit, e.g., approximately 1.1 million kg of soluble anthocyanins could be generated annually.

Decomposition of cranberry pulp and depolymerization of proanthocyanidins allows for aqueous extraction of the soluble anthocyanins for commercial use. Current methods for extraction of soluble anthocyanins from fruit waste involve chemical treatments, specialized equipment, or treatment with enzymes resulting in considerable cost to the manufacturer.

### SUMMARY

As disclosed herein, the use of specific bacteria and/or enzymes resulted in at least a 20 to 30% improvement in the production of soluble anthocyanins from fruit as compared to untreated controls. In particular, the use of a multi-organism fermentation further improves the extraction efficiency at least 23 to 69% as compared to untreated control. In one embodiment, dual microbial treatments improved anthocyanin extraction up to 435% in 24 hours and up to 10,640% in 48 hours as compared to controls. In one embodiment, anthocyanin concentrations stabilized from about 24 to about 48 hours or from about 20 to about 55 hours.

In one embodiment, a method to obtain water soluble anthocyanins is provided. In one embodiment, the method includes providing solids comprising fruit or fruit seed, skin or pulp (a “mash”, which in one embodiment is separated from juice, or “extract”); and treating the solids with one or more microbes or one or more of isolated pullulanase, isolated cellulase, isolated lipase, isolated pectinase, or isolated tannase so as to yield a mixture comprising water soluble anthocyanins. In one embodiment, the method includes providing solids comprising fruit or fruit seed, skin or pulp; and contacting the solids and one or more microbes or one or more of isolated pullulanase, isolated cellulase, isolated lipase, isolated pectinase, or isolated tannase under conditions so as to yield a mixture comprising water soluble anthocyanins. In one embodiment, the fruit is cranberry or cherry. In one embodiment, the fruit is blackberry, blueberry, grape, pomegranate, raspberry (red and black), tomato, or watermelon. In one embodiment, the solids (e.g., an extract which ma, in one embodiment, be separated from juice) are treated with pullulanase and optionally one or more other enzymes, e.g., cellulase, hemicellulase, lipase, tannase, protease, or pectinase. In one embodiment, the method does not include the use of one or more of cellulase, hemicellulase, protease, and pectinase, e.g., in the absence of pullulanase. In one embodiment, the solids are treated with one or more of microbes including Candida albicans, Saccharomyces cerevisiae, Staphylococcus lugdunesis, Klebsiella pneumoniae, Corynebacterium glutamicum, Lactobacillus plantarum, Cellulomonas cellulans, Xenorhabdus nematophilia, Pseudomonas aeruginosa, Bacillus subtilis, Bacillus cereus, Aureobasidium pullulans, or Brevibacillus laeterosporus. In one embodiment, the solids are treated with a combination of two or more microbes including Corynebacterium glutamicum, Lactobacillus plantarum, Cellulomonas cellulans, Xenorhabdus nematophilia, Pseudomonas aeruginosa, Bacillus subtilis, or Bacillus cereus. In one embodiment, the solids are treated with Corynebacterium glutamicum and Cellulomonas cellulans. In one embodiment, the solids are treated with Corynebacterium glutamicum and Xenorhabdus nematophilia. In one embodiment, the solids are treated with Cellulomonas cellulans and Xenorhabdus nematophilia. In one embodiment, the solids are treated with one or more microbes that secrete one or more of pullulanase, cellulase, lipase, pectinase, or tannase. In one embodiment, the amount of each of the microbes is about 0.5% v/v to about 1.5% v/v (the % v/v measurements are from saturated overnight cultures of the microbe grown in its optimum medium. Thus, the % is volume of culture/volume of mixture of fruit or fruit seed, skin or pulp). In one embodiment, the amount of each of the microbes is about 1.5% v/v to about 5% v/v. In one embodiment, the amount of each of the microbes is about 5% v/v to about 15% v/v. In one embodiment, the amount of water soluble anthocyanins treated with pullulanase is increased relative to water soluble anthocyanins treated with cellulase and/or pectinase. The method may further include isolating the water soluble anthocyanins from the mixture.

### BRIEF DESCRIPTION OF THE FIGURES

FIG. 1. Aqueous anthocyanin content extracted from cranberry pulp 24 hours post-enzymatic digestion.

FIG. 2. Aqueous anthocyanin content extracted from cranberry pulp 48 hours post-enzymatic digestion.

FIG. 3. Aqueous anthocyanin content extracted from cherry pulp 24 hours post-enzymatic digestion.

FIG. 4. Aqueous anthocyanin content extracted from cherry pulp 48 hours post-enzymatic digestion.

FIG. 5. Average % improvement of soluble anthocyanin content of pullulanase treated cranberry pulp vs. cellulase and pectinase treated samples.

FIG. 6. Average % improvement of soluble anthocyanin content of pullulanase treated cherry pulp vs. cellulase and pectinase treated samples.

FIG. 7. Anthocyanin content after 24 hours of incubation with increasing concentrations of select bacteria.

FIG. 8. Anthocyanin content after 48 hours of incubation with increasing concentrations of select bacteria.

FIG. 9. Anthocyanin content obtained under six different conditions and controls using Corynebacterium glutamicum and Cellulomonas cellulans. WB: Water blank, control; CRW: Corynebacterium glutamicum, control; CGB: Cellulomonas cellulans, control; Numbers 1-6 refer to experimental conditions described in Table 3.

FIG. 10. Anthocyanin content under six different conditions and controls using Corynebacterium glutamicum and Xenorhabdus nematophilia. WB: Water blank, control; CGB: Cellulomonas cellulans, control; GBP: Xenorhabdus nematophilia, control; Numbers 1-6 refer to experimental conditions described in Table 3.

FIG. 11. Anthocyanin content under six different conditions and controls using Cellulomonas cellulans and Xenorhabdus nematophilia.

FIG. 12. Concentrations of aqueous extracted anthocyanins 24 hours post-treatment. The condition producing the highest anthocyanin concentration was utilized for each experiment and control. Data represents averages of triplicates with variance between samples as error bars. Legend: CRW, Corynebacterium glutamicum; CGB, Cellulomonas celluans; GBP, Xenorhabdus nematophilia.

FIG. 13. Concentrations of aqueous extracted anthocyanins 48 hours post treatment. The condition producing the highest anthocyanin concentration was utilized for each experiment and control. Data represents averages of triplicates with variance between samples as error bars. Legend: CRW, Corynebacterium glutamicum; CGB, Cellulomonas cellulans; GBP, Xenorhabdus nematophilia.

FIG. 14. Stability of anthocyanin pigments from 24 to 48 hours. The condition producing the highest anthocyanin concentration was utilized for each experiment and control. Data represents averages of triplicates. Legend: CRW, Corynebacterium glutamicum; CGB, Cellulomonas celluans; GBP, Xenorhabdus nematophilia.

FIG. 15. Soluble anthocyanin extraction in treated vs. control cranberry pulp fermentations. Frozen, whole cranberries were pulverized and filtered to produce a solid. Cranberry pulp was transferred to sterile flasks and inoculated with a single source of bacteria or no bacteria (control). Fermentations were completed for 6 days at 30° C. with shaking. Soluble anthocyanin content was determined by centrifugal clarification and subsequent pH differential analysis of anthocyanin content (mg Cyanidin equivalence). Duplicate samples were acquired and analyzed. Range of improvement in anthocyanin extraction with microbial treatment vs. control: 23%-40%.

FIG. 16. Soluble anthocyanin extraction in dual microbe treated cranberry pulp fermentations vs. control. Legend: B: Bacillus subtilis, C: Cellulomonas sp., X: Xenorhabdus sp. Frozen, whole cranberries were pulverized and filtered to produce a solid. Cranberry pulp was transferred to sterile flasks and inoculated with a dual source of bacteria or no bacteria (control). Fermentations were completed for one day at 30° C. with shaking. Soluble anthocyanin content was determined by centrifugal clarification and subsequent pH differential analysis of anthocyanin content (mg Cyanidin equivalence). Duplicate samples were acquired and analyzed. Range of improvement in anthocyanin extraction of microbial treatment: 23-69%.

### DETAILED DESCRIPTION

Of naturally-derived antioxidants, polyphenolic compounds have significant antioxidant potential and various medicinal and industrial applications (Foti, 2007). Specifically, anthocyanins are bioactive polyphenolic compounds commonly found in fruit and flowers that have demonstrated antioxidant, anti-cancer, and cardioprotective properties (He & Giusti, 2010). Thus, these compounds have a variety of medicinal applications and are of interest to the supplement, fine chemical, and pharmaceutical industries. While fruits such as berries typically contain high concentrations of anthocyanins, 30-40% remain trapped in the skin and seeds in the form of membrane and cell wall complexes or polymerized networks of anthocyanins (e.g., proanthocyanidins) (White et al., 2011). As such, waste from fruit processing (e.g., pomace) serves as a unique source of untapped anthocyanins. However, while waste from fruit processing contains substantial residual anthocyanin content, current methods for extracting these high value compounds require significant initial and ongoing investment.

Various techniques exist for extracting anthocyanins from pomace. These technologies fit into three categories: mechanical (Tournay &Tournay, 2012; Ablett, 2009; Mazza & Pronyk, 2015), chemical (Ablett, 2009; Howard et al., 2012; Philip, 1976), and enzymatic (Chrikhande, 1984). Mechanical methods use ultrasonication (He et al., 2016; Ghafoor et al., 2009), microwave extraction, or other extraction-specific instrumentation. Each requires specialized equipment, with limited throughput, which results in high start-up costs and may require specialized workforce training. Chemical methods for anthocyanin extraction have continued high operating costs, and many chemical methods result in residual contamination that must be removed prior to use in the food and supplement industry.

The present disclosure relates to the use of specific microbes and/or enzymes to facilitate the low-cost and renewable extraction of anthocyanins from, for example, fruit waste generated from, for example, the seeds or skins of cranberries, grapes and cherries. For instance, anthocyanin extraction can be increased by at least 23-69% using certain bacteria. This method provides an easily adoptable method for existing fruit processors to utilize waste in a growing secondary market.

In one embodiment, the method utilizes specific microbes and microbial mixtures to generate the enzymes required to free anthocyanins from the fruit waste matrix. Microbial fermentation of pomace used to generate high value compounds is not unheard of but has not previously been applied to this specific problem. Instead, microbial fermentation is currently used to extract tannic and tartaric acids, essential oils and flavorants, and various macromolecules (e.g., proteins and oils) from plant waste. Additionally, microbes have previously been used to generate pigments and antioxidants for the feedstock industry, however, these are largely facilitated by genetically modified organisms. In one embodiment, the method employs individual or select combinations of enzymes and/or microorganisms, e.g., unaltered or native, i.e., non-recombinant microorganisms, to degrade pomace and release soluble anthocyanin compounds, e.g., for use in the supplement, fine chemical, and pharmaceutical industries.

As disclosed herein, certain enzymes and/or microorganisms allow for successful enzymatic methods for extracting anthocyanins from, for example, fruit matrixes. The fruit waste matrix consists of membranes, complex carbohydrates (e.g. pectin, pullulan, cellulose), and anthocyanin conjugates, and proanthocyanidins, and decomposition of the waste matrix allows for aqueous extraction of the soluble anthocyanins. In one embodiment, the method employs one or more of cellulase, pullulanase, pectinase, lipase, and/or tannase. In one embodiment, the method employs bacteria that express one or more of cellulase, pullulanase, pectinase, lipase, or tannase. In one embodiment, the method employs one or more acid secreting microbes, e.g., to stabilize anthocyanin monomers post-extraction. In one embodiment, the enzymes are secreted from or extracted from the microorganisms that are employed in the method are shown in Table 1. Microbial sources are inexpensive, renewable, and in many cases, readily in use in the food industry.

This disclosure provides at least one method to enable extraction of residual soluble anthocyanins from a waste product into a commercially viable product. Soluble anthocyanins can be utilized for production of human supplements, food ingredients, dyes, cosmetics, antioxidants, and fine chemicals. Anthocyanins have been studied extensively for their bioactivity. Wisconsin cranberry processors generate millions of pounds of cranberry fruit waste annually. This technique allows them to monetize a waste product. Additionally, as this technique works on cherry fruit it is reasonable that it might work on other fruits and flowers that are rich in anthocyanins, generating a variety of anthocyanin products for downstream applications. Thus, this technique allows fruit processors to monetize a waste product. Additionally, it is reasonable that other fruits and flowers that are rich in anthocyanins may be subjected to the method, generating a variety of anthocyanin products for downstream applications.

Currently anthocyanins are harvested from the soluble extracts of natural sources, such as juice and whole fruit. Whole fruit and juices have other commercial applications, namely human consumption, which drives up the cost of producing anthocyanin-rich components. Additionally, fruit processors typically utilize purified enzymes in their current processing schemes, which indicates that addition of another purified enzyme is not an arduous task and could be easily adapted into the current food and waste processing streams. Once extracted, soluble anthocyanins can be utilized for production of human supplements, food ingredients, dyes, cosmetics antioxidants, and fine chemicals.

While other technologies exist for extracting anthocyanins from fruit solids, each has a significant cost associated with it. For example, high pressure steam extraction requires specialized equipment and personnel. Chemical extraction has ongoing consumable reagent costs and, additionally, some chemical extraction techniques cannot be used for food ingredient and supplement applications as they involve reagents not fit for human consumption. Enzymatic extraction also has considerable ongoing consumable reagent costs which are typically much higher than even chemical extraction costs. This disclosure provides methods that harness the efficacy of enzymatic extraction with the cost-savings of using microbes as a biorenewable generator of enzymes.

Cranberry pulp contains membranes, complex carbohydrates (e.g. pectin, pullulan, cellulose), proteins, anthocyanin conjugates, and proanthocyanidins (e.g. polymeric anthocyanins). Decomposition of cranberry pulp and depolymerization of proanthocyanidins allows for aqueous extraction of the soluble anthocyanins for commercial use. Current methods for extraction of soluble anthocyanins from fruit waste involve chemical treatments, specialized equipment, or treatment with enzymes resulting in considerable cost to the manufacturer. However, multiple bacterial species secrete enzymes that can degrade components of the cranberry pulp, and potentially release soluble anthocyanins. Use of food grade, fermentative bacteria to degrade the pulp may allow for production of soluble anthocyanins that feed into existing anthocyanin production pipelines.

When working with anthocyanins, it is important to note that they are unstable in aqueous environments. Anthocyanins act as scavengers of free radicals, and upon reaction they photobleach to become colorless to the human eye and to the instrumentation used in this work. In addition to increasing extraction of anthocyanins from the pulp, stabilization of the free anthocyanins is desirable. Traditional stabilization is achieved by acidification of the anthocyanins, which results in both greater color and longer retention of structure in aqueous solutions. Bacteria that secrete acids may also be useful in improving yield from extractions.

This disclosure describes a method by which cranberry solids are treated with a variety of bacteria resulting in an increase of soluble anthocyanins overtime.

### Exemplary Method for Anthocyanin Production using Enzymes

In one embodiment, this disclosure describes a method by which fruit solids (pulp) such as cranberry or cherry solids are treated with the enzyme pullulanase, resulting in a significant increase in soluble anthocyanins as compared to other enzymes and untreated fruit pulp. Pullulanase (EC 3.2.1.41, limit dextrinase, amylopectin 6-glucanohydrolase, bacterial debranching enzyme, debranching enzyme, alpha-dextrin endo-1,6-alpha-glucosidase, R-enzyme, pullulan alpha-1,6-glucanohydrolase) is a glucanase, an amylolytic exoenzyme, that degrades pullulan. Specifically:

1) 24 hours of incubation with 0.5-100 U pullulanase resulted in an average of 667% (range 110%-2300%) increase in anthocyanins extracted from 5 g samples of cranberry pulp as compared to pectinase extraction. After 48 hours, this decreased to 611% (range: 110-1780%) presumably due to degradation of soluble anthocyanins in the aqueous media.

2) 24 hours of incubation with 0.5-100 U pullulanase resulted in an average of 360% (range 67%-1278%) increase in anthocyanins extracted from 5 g samples of cranberry pulp as compared to cellulase extraction. After 48 hours, this decreased to 241% (range: 65-593%) presumably due to degradation of soluble anthocyanins in the aqueous media.

3) 24 hours of incubation with 0.5-100 U pullulanase resulted in an average of 391% (range 200-1385%) increase in anthocyanins extracted from 5 g samples of cherry pulp as compared to pectinase extraction. After 48 hours, pectinase digestion resulted in no detectable soluble anthocyanins, making comparison at this timepoint impossible.

4) 24 hours of incubation with 0.5-100 U pullulanase resulted in an average of 128% (range 69%-202%) increase in anthocyanins extracted from 5 g samples of cherry pulp as compared to cellulase extraction. After 48 hours, this value increased, though this may be an artifact of the degradation of anthocyanins in the cellulase samples.

### Exemplary Method for Anthocyanin Production using Bacteria

Cranberry pulp was treated with one of fourteen unique bacteria or fungi. A list of exemplary bacteria and fungi is found below.

1) Treatment of 5 g of cranberry pulp with the following bacteria or fungi resulted in increases in anthocyanin content as compared to unfermented control after 24 hours: Xenorhabdus nematophilia, Corynebacterium g/utamicum, Cellulomonas cellulans, Aureobasidium pullulans, Pseudomonas aeruginosa, Staphylococcus lugdunesis, Lactobacillus plantarum, Klebsiella pneumonieae, Candida albicans, Saccharomyces cerevisieae, Brevibacillus laetosporus, Bacillus cereus, and Bacillus subtilis.

2) Treatment of 5 g of cranberry pulp with the following bacteria or fungi resulted in increases in anthocyanin content as compared to unfermented control after 48 hours: Xenorhabdus nematophilia, Corynebacterium glutamicum, Cellulomonas cellulans, Lactobacillus plantarum, Klebsiella pneumonieae, Pseudomonas aeruginosa, Bacillus cereus, and Bacillus subtilis.

3) Treatment of 5 g of cranberry pulp with the following bacteria or fungi resulted in increases in soluble anthocyanin content from 24 to 48 hours: Corynebacterium glutamicum and Bacillus subtilis.

4) Treatment of 5 g of cranberry pulp with the following bacteria or fungi resulted in less than 10% reduction of soluble anthocyanins from 24 to 48 hours, as compared to a non-innoculated blank that suffered 17% loss of soluble anthocyanins: Xenorhabdus nematophilia, Corynebacterium glutamicum, and Cellulomonas cellulans.

5) Treatment of 5 g of cranberry pulp with increasing concentrations of Bacillus cerus, Bacillus subtilis, Brevibacillus laeterosporus, and Cellulmonas cellulans resulted in decreasing concentrations of soluble anthocyanins to the point that they contain less soluble anthocyanins than a comparable untreated sample after 24-48 hours. This indicates these bacteria degrade anthocyanins at high concentrations.

6) Treatment of 5g of cranberry pulp with increasing concentrations of Xenorhabdus nematophilia and Corynebacterium glutamicum did not exhibit microbe-concentration dependent increases in soluble anthocyanin content. However, all concentrations of these two microbes produced soluble anthocyanins at a concentration higher than the water blank.

In comparing results from the use of isolated enzyme(s) to the use of microbe(s), at peak extraction, in one embodiment, enzyme-assisted extraction resulted in 130% more anthocyanins than control after 24 hours, while microbial extraction resulted in 10,640% more anthocyanins than control after 24 hours. Enzyme assisted extraction demonstrated maximal efficiency at concentrations between 5 to 100 U/mL applied to 5 grams of cranberry extract. Maximal extraction resulted in aqueous anthocyanin concentrations of approximately 0.19 mg of soluble anthocyanins per gram of cranberry pulp and a maximum of 0.19 mg of soluble anthocyanins per unit of enzyme utilized. For example, with a cost of $0.002 per enzyme unit, the use of enzymes for anthocyanin extraction would cost $2 per kg of cranberry pulp and result in 95 g of anthocyanins per dollar. The use of two microbes for anthocyanin extraction can be obtained for less than $600.00 and cultured for less than $1.00 per liter. The maximal aqueous extraction of anthocyanins resulted in 0.35 mg of anthocyanins per g of cranberry pulp with treatment of 0.5-2% of each microbe. The initial kg of cranberry pulp would cost $601 to treat using this method and yield 350 g of soluble anthocyanins. This is 58 g of anthocyanins per dollar. However, the next kg, and all following kg of cranberry pulp will have a cost of $1.00 to process using this method, resulting in a yield of 350 g of anthocyanins per dollar. One processing facility reported generating 2 million lbs, or 907,185 kg, of cranberry pulp annually. Using the enzyme-extraction method, this can result in 172,365 g of soluble anthocyanins and cost $1,814,370.00. Using the microbial extraction method, this is anticipated to result in 317,515 g of soluble anthocyanins at a cost of $907,785 00, or 368% more anthocyanins per dollar than the enzyme extraction method.

This invention will be described by the following non-limiting examples.

### EXAMPLE 1

### Methods

Preparation of pulp: Two and a half (2.5) pounds of whole frozen cranberries were soaked in 4 liters of 3% bleach solution for 10 minutes to kill surface microbes. Cranberries were rinsed three times with fresh de-ionized water and broken cranberries were removed. Cranberries were strained prior to juicing with a Breville centrifugal juicer. Pulp was collected and juiced again to remove excess moisture. Pulp was collected and stored at −20° C. Cherry fruit was treated in the same manner.

Enzyme challenge: Enzymes were purchased from Sigma-Aldrich. Microbial pullulanase (about 400 U/mL) was procured as a liquid and measured directly. Cellulase and pectinase were dissolved in 50 mL of ice cold water to a concentration of 300 U/mL. Cranberry or cherry pulp was thawed at room temperature. Five grams of each pulp was measured into separate 15 mL sterile conical tubes. Final enzyme concentrations of 0.5 U/mL to 100 U/mL were generated by addition of the appropriate amount of liquid enzyme to the tube and addition of cold water to a final volume of 10 mL. Blank tubes contained water and pulp with no additional enzyme. Tubes were gently agitated for 48 hours at 100 rpm on a temperature controlled orbital shaker at 100 rpm to simulate stirring action. Samples were taken at 0, 24, and 48 hours by separation of the liquid phase from the solids by brief centrifugation at 1500 rpm for 10 minutes. One milliliter of liquid was collected, and the tubes were vortexed to resuspend pelleted solids prior to return to shaking for the duration of the experiment.

Determination of lambda maximum for anthocyanin quantification: Anthocyanin content can vary by fruit varietal and strain, and the wavelength of maximum absorption (lambda max) varies by the anthocyanins present in the fruit. As it was not possible to know the exact varietal represented by the frozen fruit stock, the lambda max was experimentally determined by spectrophotometric analysis of the aqueous extract of the fruit as described by Lee et. al. (2005). Aqueous extracts of blank samples were collected prior to incubation (e.g., 0 hr of enzyme challenge) and diluted 1/10 with 25 mM KCl solution (pH 1.0). Absorbance was measured from 450-750 nm in 5 nm increments. A separate aqueous extract was diluted 1/10 with 0.04 M Sodium acetate buffer (pH 4.5) and absorbance was measured from 450-750 nM in 5 nM increments. Absorbance at pH 4.5 was subtracted from absorbance at pH 1.0. Lamda max is the wavelength of maximum absorbance after subtraction. For cranberry, this maximum was found to be 520 nm, which corresponds to the major anthocyanin Cyanidin and its soluble glycoconjugates. For cherry, this maximum was found to be 515 nm, which corresponds to Malvidin and its soluble glycoconjugates.

pH differential assay of anthocyanin quantification: Anthocyanin quantification was performed spectrophotometrically using the method described by Lee et. al. (2005). Briefly, 0.1 mL of aqueous anthocyanin extract was diluted in 0.9 mL of pH 1.0 solution described previously. A separate sample of 0.1 mL of aqueous anthocyanin extract was diluted into 0.9 mL of pH 4.5 solution described previously. The samples were both read at the lambda maximum for the fruit sample as described previously and 700 nm. Anthocyanin content was calculated by subtracting the 700 nm reading from the lambda max at each pH as a background correction and then further subtracting the corrected pH 4.5 reading from the pH 1.0 reading, resulting in a single absorbance value. Absorbance was then converted to mg of major anthocyanin (cyanidin glucoside for cranberry and malvidin glucoside for cherry, respectively) using Lambert-Beer's Law. Finally, one mg of major anthocyanin was divided by the initial mass of fruit pulp, resulting in a measurement of mg/g anthocyanins.

### Results

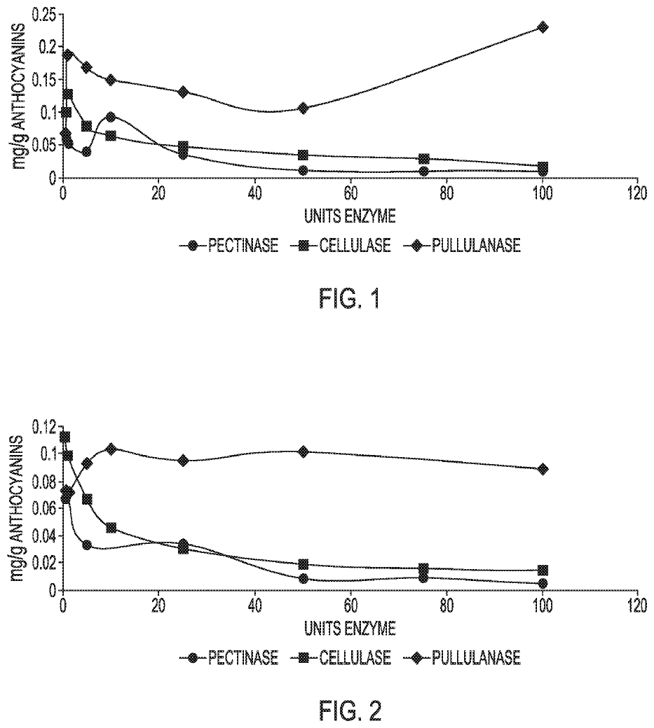

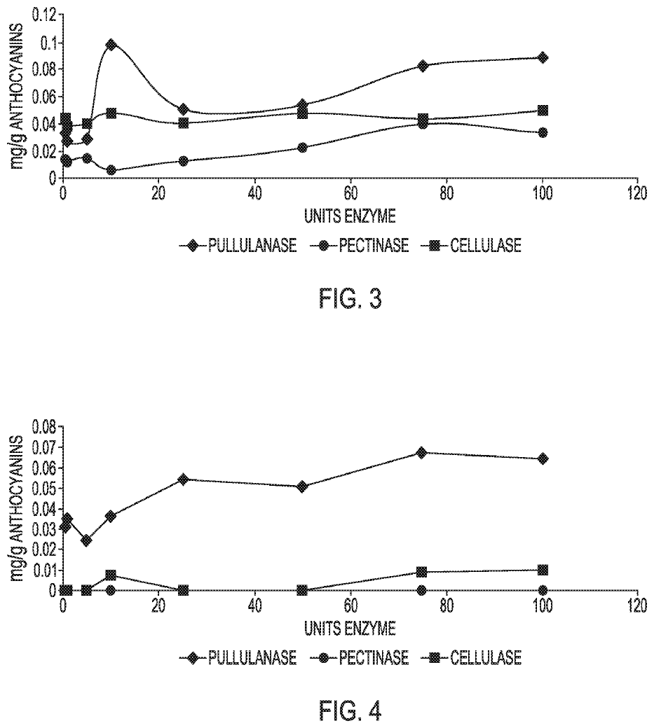

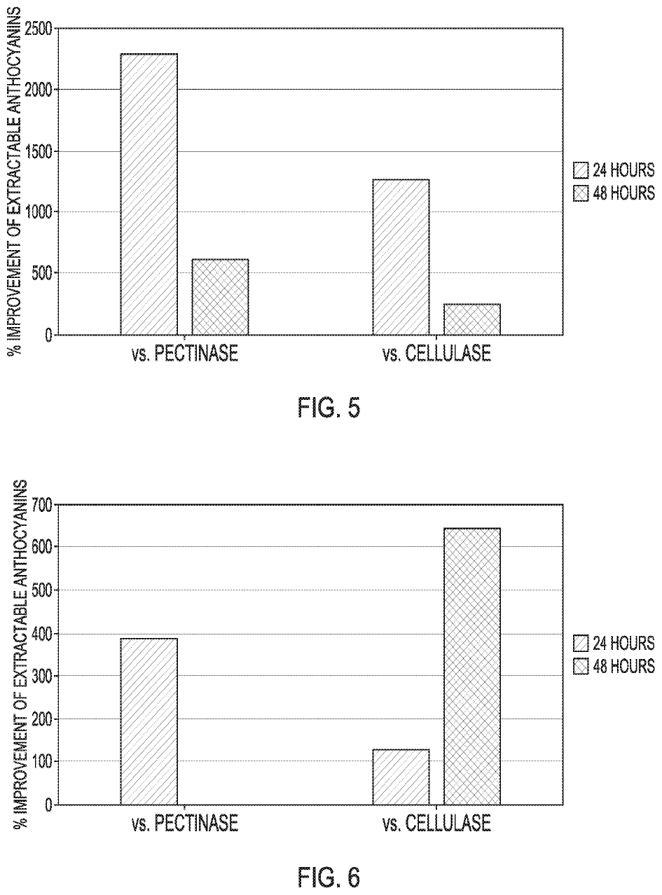

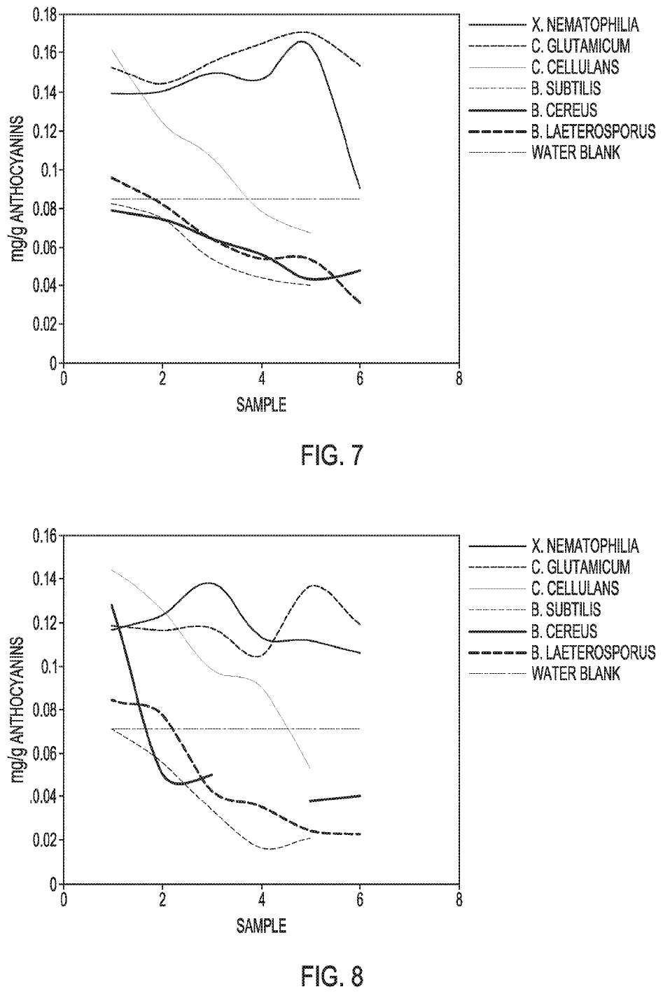

Pullulanase enzymatic extraction was compared to enzymatic extractions using cellulase and pectinase. Eight enzyme concentrations were assayed in duplicate and the anthocyanin concentration of aqueous extracts was quantified at 24 and 48 hours (FIGS. 1-4). Average anthocyanin content for duplicates was plotted against enzyme concentration. Note: when no datapoint is presented, duplicate measurements were not available.

At 7/8 concentrations, the aqueous extract of cranberry pulp after pullulanase treatment resulted in higher anthocyanin concentrations than pectinase and cellulase treatments after both 24 and 48 hours. Notably, increasing concentrations of cellulase and pectinase resulted in degradation of anthocyanin content that accelerated from 24-48 hours.

After 24 hours of enzymatic digestion, 5/8 concentrations of pullulanase outperformed cellulase and 8/8 concentrations of pullulanase outperformed pectinase to produce soluble anthocyanins. After 48 hours, pullulanase outperformed both enzymes at all concentrations. All pectinase samples failed to show any anthocyanin content after 48 hours.

Overall, pullulanase digestion resulted in a greater average concentration of soluble anthocyanins at both 24-hour and 48-hour timepoints. Due to the complete degradation of pectinase treated samples after 48 hours, no data is provided. Similarly, several cellulase treated samples were completely degraded after 48 hours, and the 48-hour data is representative of the average of three data points. See FIG. 4.

### EXAMPLE 2

Multiple bacterial species secrete enzymes that can degrade components of cranberry pulp, and potentially release soluble anthocyanins. Use of food grade, fermentative bacteria to degrade the pulp may allow for production of soluble anthocyanins that feed into existing anthocyanin production pipelines.

### Methods

Preparation of pulp: Two and a half (2.5) pounds of whole frozen cranberries were soaked in 4 liters of 3% bleach solution for 10 minutes to kill surface microbes. Cranberries were rinsed three times with fresh de-ionized water and broken cranberries were removed. Cranberries were strained prior to juicing with a Breville centrifugal juicer. Pulp was collected and juiced again to remove excess moisture. Pulp was collected and stored at −20° C.

Preparation of microbial stocks: Microbial glycerol stocks, Kwik-Stix, or Lyfo-Disks were acquired from American Type Culture Collection, VWR, the UW-Parkside Biology Department, or the generous gift of Greg Richards, Ph.D.

TABLE 1
Bacterial cultures used in this study

| | | Optimal growth |
|---|---|---|
| Microbe | Media | temperature |
| Acetobacter acetii | Acetobacter medium | 25 |
| Bacillus cereus | LB | 30 |
| Bacillus subtilis | LB | 30 |
| Bifidobacterium bifidum | LB | 37 |
| Brevibacillus laeterosporus | TSB | 37 |
| Candida albicans | YM | 37 |
| Cellulomonas cellulans | PTYG | 25 |
| Corynebacterium glutamicum | LB | 37 |
| Klebsiella pneumoniae | LB | 37 |
| Lactobacillus plantarum | TSB | 37 |
| Pseudomonas aeruginosa | TSB | 37 |
| Saccharomyces cereviseae | YM | 25 |
| Staphylococcus lugdunesis | TSB | 25 |
| Xenorhabdus nematophilia | LB | 25 |

Microbes were grown to OD₆₀₀ in optimal media at optimal temperature and centrifuged to pellet cells. Glycerol stocks were generated by addition of 50% glycerol to the pellet of 1 mL of media. Glycerol stocks were kept at −80° C. until use and were not refrozen after use.

Glycerol stocks were used to inoculate 1 L of sterile optimal media. Microbes were grown at optimal growth temperature with shaking when applicable until saturation of the media was achieved. Microbes were pelleted via centrifugation and frozen at −80° C. until use.

Inoculation of cranberry pulp and collection of samples for anthocyanin quantification: 5 g of prepared cranberry pulp was aseptically transferred into sterile 50 mL conical tubes for study. Microbial stocks were thawed and resuspended in 200 mL of sterile water. 10 mL of bacterial resuspension was transferred into the conical tube followed by 10 mL of sterile water, resulting in a final liquid volume of 20 mL. Experiments were conducted in triplicate. Initially, pulp was resuspended by inversion, and 1 mL of liquid was collected and frozen to provide reference concentration at 0 hours of incubation. Tubes were placed horizontally on a incubating shaker and shaken at 25° C. and 100 rpm for the duration of the experiment. After 24 and 48 hours, tubes were centrifuged at 1000 rpm for 5 minutes to sediment pulp and 1 mL of liquid was collected. Additionally, 100 μL of liquid was used to inoculate an agar plate made of the appropriate media for the microbe, with the exception of Aureobasidium pullulans, which was inoculated onto plates by sterile swab. Plates were incubated at optimal temperature for microbial growth for 24-48 hours and colonies were counted. Liquid fractions were frozen until anthocyanin quantification was performed. Uninoculated samples of cranberry pulp were prepared similarly, replacing 10 mL of microbial culture with 10 mL of sterile water.

Determination of lambda maximum for anthocyanin quantification: Anthocyanin content can vary by fruit varietal and strain, and the wavelength of maximum absorption (lambda max) varies by the anthocyanins present in the fruit. As it was not possible to know the exact varietal represented by the frozen fruit stock, the lambda max was experimentally determined by spectrophotometric analysis of the aqueous extract of the fruit as described by Lee et al. (2005). Aqueous extracts of blank samples described earlier were collected prior to incubation (e.g., 0 hour of enzyme challenge) and diluted 1/10 with 25 mM KCl solution (pH 1.0). Absorbance was measured from 450-750 nm in 5 nm increments. A separate aqueous extract was diluted 1/10 with 0.04 M Sodium acetate buffer (pH 4.5) and absorbance was measured from 450-750 nM in 5 nM increments. Absorbance at pH 4.5 was subtracted from absorbance at pH 1.0. Lamda max is the wavelength of maximum absorbance after subtraction. For cranberry, this maximum was found to be 520 nm, which corresponds to the major anthocyanin cyanidin and its soluble glycoconjugates.

pH differential assay of anthocyanin quantification: Anthocyanin quantification was performed spectrophotometrically using the method described by Lee et. al. (2005). Briefly, 0.1 mL of aqueous anthocyanin extract was diluted in 0.9 mL of pH 1.0 solution described previously. A separate sample of 0.1 mL of aqueous anthocyanin extract was diluted into 0.9 mL of pH 4.5 solution described previously. The samples were both read at the lambda maximum for the fruit sample as described previously and 700 nm. Anthocyanin content was calculated by subtracting the 700 nm reading from the lambda max at each pH as a background correction and then further subtracting the corrected pH 4.5 reading from the pH 1.0 reading, resulting in a single absorbance value. Absorbance was then converted to mg of major anthocyanin (cyanidin glucoside for cranberry and malvidin glucoside for cherry, respectively) using Lambert-Beer's Law. Finally, mg of major anthocyanin was divided by the initial mass of fruit pulp, resulting in a measurement of mg/g anthocyanins.

Effect of varying amounts of microbial inoculation on anthocyanin extraction. Increasing amounts of bacteria were utilized to degrade 5 g of cranberry pulp. Bacteria were inoculated into optimal growth media using glycerol stocks and grown to saturation at optimal temperature. The growth from 1 L of culture was harvested via centrifugation. The equivalent of 25, 50, 100, 150, 200, or 300 mL of bacterial growth was transferred to 50 mL sterile conical tubes containing cranberry pulp. The bacteria was resuspended in 20 mL of sterile deionized water. Timepoints were taken at 0 hours, 24 hours, 48 hours as described previously. Bacterial growth was confirmed via plating. Anthocyanin content was quantified via the pH differential method.

### Results

Experiment: Inoculation of triplicate samples of 5 g of prepared cranberry pulp with identical concentrations of microbes over 48 hour time course. Aliquots of soluble anthocyanins were collected after 24 and 48 hours and quantified via the pH differential method.

TABLE 2
Average anthocyanin content of microbe treated cranberry pulp
after 24 and 48 hours. Data represents average of three replicates,
except in the case of ‘Blank’ samples in which the average
of four replicates is used. Where ‘nd’ is indicated,
samples were either below the limit of detection for the assay
or turbidity of the samples prevented further analysis.

| | Average | Average |
|---|---|---|
| | anthocyanins at | anthocyanins at |
| Microbe | 24 hours (mg/g) | 48 hours (mg/g) |
| Candida albicans | 0.151 | 0.062 |
| Saccharomyces cerevisiae | 0.13 | 0.055 |
| Staphylococcus lugdunesis | 0.147 | 0.057 |
| Klebsiella pneumoniae | 0.147 | 0.101 |
| Corynebacterium glutamicum | 0.106 | 0.236 |
| Lactobacillus plantarum | 0.099 | 0.08 |
| Cellulomonas cellulans | 0.149 | 0.137 |
| Xenorhabdus nematophilia | 0.145 | 0.142 |
| Pseudomonas aeruginosa | 0.148 | 0.139 |
| Bacillus subtilis | 0.141 | 0.143 |
| Bacillus cereus | 0.24 | 0.076 |
| Aureobasidium pullulans | 0.178 | nd |
| Brevibacillus laeterosporus | 0.153 | nd |
| Blank* | 0.085 | 0.071 |

### Conclusions

Anthocyanin content increased in the presence of all tested microbes after 24 hours. This is expected as anthocyanin aglycones are lipid soluble, and any deglycosylated anthocyanins would be more soluble in bacteria-rich solutions than bacteria-free solutions. The relative concentration of anthocyanins after 24 hours ranged from 116-280% of average water blanks.

Anthocyanin content decreased in the presence of some microbes after 48 hours. As all microbes showed continued growth from 24-48 hours upon plating, this is likely due to metabolic degradation of the anthocyanins, or at least failure to extract more anthocyanins than the natural rate of photobleaching degrades. Specifically, the fungi Candida albicans, Saccharomyces cerevisiae, Aureobasidium pullulans, and the bacteria Staphylococcus lugdunesis, and Brevibacillus laeterosporus all demonstrated decreases in anthocyanin content to below water blank (e.g., photobleaching) levels after 48 hours.

Anthocyanin content increased in the presence of some microbes after 48 hours, which indicates some bacteria are able to stabilize anthocyanins against photobleaching and/or the level of extraction of soluble anthocyanins is higher than the degradation of freed anthocyanins. Specifically, Corynebacterium glutamicum and Bacillus subtilis demonstrated increases in anthocyanin content from 24 to 48 hours. Cellulomonas cellulans, Xenorhabdus nematophilia, and Pseudomonas aeruginosa all demonstrated less than 10% loss of anthocyanins, which is well below the 17% loss observed in the water blanks.

Additional analyses indicated that collection of non-solid (aqueous) material at 48 hours, rather than at later time points, resulted in the maximal extraction to degradation ratio. Extracted anthocyanins can be stored without degradation at −20° C. indefinitely. Purification may be achieved by a variety of methods including but not limited to ion exchange chromatography or reverse phase HPLC on C18 columns.

Experiment: Inoculation of triplicate samples of 5 g of prepared cranberry pulp with increasing concentrations of microbes over 48 hour time course. Aliquots of soluble anthocyanins were collected after 24 and 48 hours and quantified via the pH differential method.

The equivalent of 25, 50, 100, 150, 200, or 300 mL of bacterial culture were concentrated in 50 mL culture tubes and incubated with 5 g of cranberry pulp and 20 mL sterile water for 24 hours.

The equivalent of 25, 50, 100, 150, 200, or 300 mL of bacterial culture were concentrated in 50 mL culture tubes and incubated with 5 g of cranberry pulp and 20 mL sterile water for 48 hours.

### Conclusions

1. Increasing bacterial concentration relative to cranberry pulp does not always result in extraction of more anthocyanins.

2. Bacterial concentration does not appear to affect the effectiveness of X. nematophilia and C. glutamicum.

3. C. cellulans, B. subtilis, B. cereus, and B. laeterosporus cause degradation of soluble anthocyanins at high concentrations, e.g., about 3% v/v to about 6% v/v. At lower concentrations, freeing of anthocyanins was higher than or equal to water blank for all bacteria.

### EXAMPLE 3

### Methods

Preparation of pulp: Two and a half (2.5) pounds of whole frozen cranberries were soaked in 4 liters of 3% bleach solution for 10 minutes to kill surface microbes. Cranberries were rinsed three times with fresh de-ionized water and broken cranberries were removed. Cranberries were strained prior to juicing with a Breville centrifugal juicer. Pulp was collected and juiced again to remove excess moisture. Pulp was collected and stored at −20° C.

Preparation of microbial stocks: Microbial glycerol stocks, Kwik-Stix, or Lyfo-Disks were acquired from American Type Culture Collection, VWR, the UW-Parkside Biology Department, or the generous gift of Greg Richards, Ph.D.

TABLE 3
Bacterial cultures used in this study

| | | Optimal growth |
|---|---|---|
| Microbe | Media | temperature |
| Cellulomonas cellulans | PTYG | 25 |
| Corynebacterium glutamicum | LB | 37 |
| Xenorhabdus nematophilia | LB | 25 |

Microbes were grown to OD₆₀₀ in optimal media at optimal temperature and centrifuged to pellet cells. Glycerol stocks were generated by addition of 50% glycerol to the pellet of 1 mL of media. Glycerol stocks were kept at −80° C. until use and were not refrozen after use.

Glycerol stocks were used to inoculate 1 L of sterile optimal media. Microbes were grown at optimal growth temperature when applicable until saturation of the media was achieved. Microbes were pelleted via centrifugation and frozen at −80° C. until use.

Inoculation of cranberry pulp and collection of samples for anthocyanin quantification: 5 g of prepared cranberry pulp was aseptically transferred into sterile 50 mL conical tubes for study. Microbial stocks were thawed and resuspended in 100 mL of sterile water. The bacterial resuspension was inoculated into 1100 mL of sterile water (stock 1) and mixed to homogenize. Stock 1 was then 10-fold diluted into water to generate Stock 2. 1-7.5 mL of Stock 2 or 1-5 mL of Stock 1 were inoculated into the conical tube followed by enough sterile water to generate a total liquid volume of 40 mL. Experiments were conducted in triplicate. Initially, pulp was resuspended by inversion, and 1 mL of liquid was collected and frozen to provide reference concentration at 0 hours of incubation. Tubes were placed horizontally on an incubating shaker and shaken at 25° C. and 100 rpm for the duration of the experiment. After 24 and 48 hours, tubes were rested for 10 minutes on a flat surface to allow sedimentation of the remaining pulp. At each timepoint, 1 mL of liquid was retained for experimentation. Liquid fractions were frozen until anthocyanin quantification was performed. Uninoculated samples of cranberry pulp were prepared similarly by replacing 40 mL of diluted culture with 40 mL of sterile water.

Determination of lambda maximum for anthocyanin quantification: Anthocyanin content can vary by fruit varietal and strain, and the wavelength of maximum absorption (lambda max) varies by the anthocyanins present in the fruit. As it was not possible to know the exact varietal represented by the frozen fruit stock, the lambda max was experimentally determined by spectrophotometric analysis of the aqueous extract of the fruit as described by Lee et al. (2005). Aqueous extracts of blank samples described earlier were collected prior to incubation (e.g., 0 hr of enzyme challenge) and diluted 1/10 with 25 mM KCl solution (pH 1.0). Absorbance was measured from 450-750 nm in 5 nm increments. A separate aqueous extract was diluted 1/10 with 0.04 M sodium acetate buffer (pH 4.5) and absorbance was measured from 450-750 nM in 5 nM increments. Absorbance at pH 4.5 was subtracted from absorbance at pH 1.0. Lambda max is the wavelength of maximum absorbance after subtraction. For cranberry, this maximum was found to be 520 nm, which corresponds to the major anthocyanin cyanidin and its soluble glycoconjugates.

pH differential assay of anthocyanin quantification: Anthocyanin quantification was performed spectrophotometrically using the method described by Lee et. al. (2005). Briefly, 0.1 mL of aqueous anthocyanin extract was diluted in 0.9 mL of pH 1.0 solution described previously. A separate sample of 0.1 mL of aqueous anthocyanin extract was diluted into 0.9 mL of pH 4.5 solution described previously. The samples were both read at the lambda maximum for the fruit sample as described previously and 700 nm. Anthocyanin content was calculated by subtracting the 700 nm reading from the lambda max at each pH as a background correction and then further subtracting the corrected pH 4.5 reading from the pH 1.0 reading, resulting in a single absorbance value. Absorbance was then converted to mg of major anthocyanin (cyanidin glucoside for cranberry) using Lambert-Beer's Law. Finally, mg of major anthocyanin was divided by the initial mass of fruit pulp, resulting in a measurement of mg/g anthocyanins.

Experiment 1: Proportionally increasing concentrations of two bacteria were inoculated into 5 g of cranberry pulp as described above. Time points were collected a 0, 24, and 48 hr and quantified via the pH differential method.

Experiment 1 a: Corynebacterium glutamicum+Cellulomonas cellulans
Experiment 1b: Corynebacterium glutamicum+Xenorhabdus nematophilia
Experiment 1 c: Cellulomonas cellulans+Xenorhabdus nematophilia

TABLE 4
Experiment 1 bacterial concentrations

| | Bacteria 1 | Bacteria 2 |
|---|---|---|
| Condition # | concentration (v/v) | concentration (v/v) |
| 1 | 0.2% | 0.2% |
| 2 | 0.5% | 0.5% |
| 3 | 1% | 1% |
| 4 | 1.5% | 1.5% |
| 5 | 2% | 2% |
| 6 | 10% | 10% |

Final calculation of extraction efficiency: To quantify extraction efficiency as compared to control, the following calculation was used:

$$= \frac{( C {E}_{t} - C {E}_{0} )}{( C {B}_{t} - C {B}_{0} )} \times 100$$

Where CE=concentration of experimental aqueous anthocyanin

extraction, t=time point, 0=time zero

And CB=concentration of control aqueous anthocyanin extraction

### Results

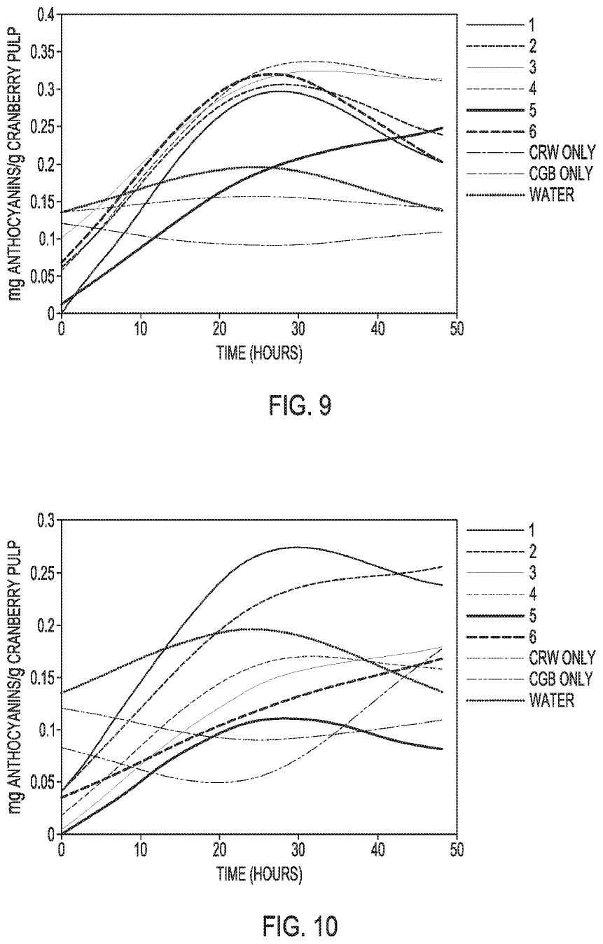

### Conclusions for FIG. 9:

1) All concentrations of bacteria improved aqueous extraction of anthocyanins from time-point 0 hr to 24 hr.
2) All experimental conditions improved aqueous extraction of anthocyanins as compared to water or single bacteria controls after both 24 and 48 hours.
3) Conditions 3-5 resulted in retention of anthocyanins from 24 to 48 hrs.

### Conclusions for FIG. 10:

1) All concentrations of bacteria improved aqueous extraction of anthocyanins from time-point 0 hr to 24 hr.
2) Conditions 1 and 2 resulted in higher anthocyanin extraction after both 24 and 48 hours than single bacteria or water-based controls.
3) All experimental conditions resulted in less overall anthocyanin extraction than Experiment 1a.

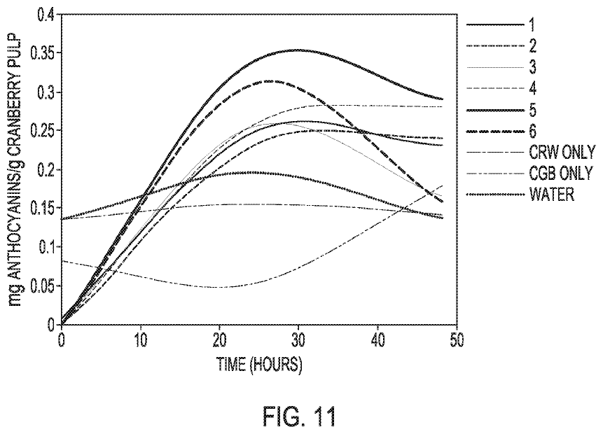

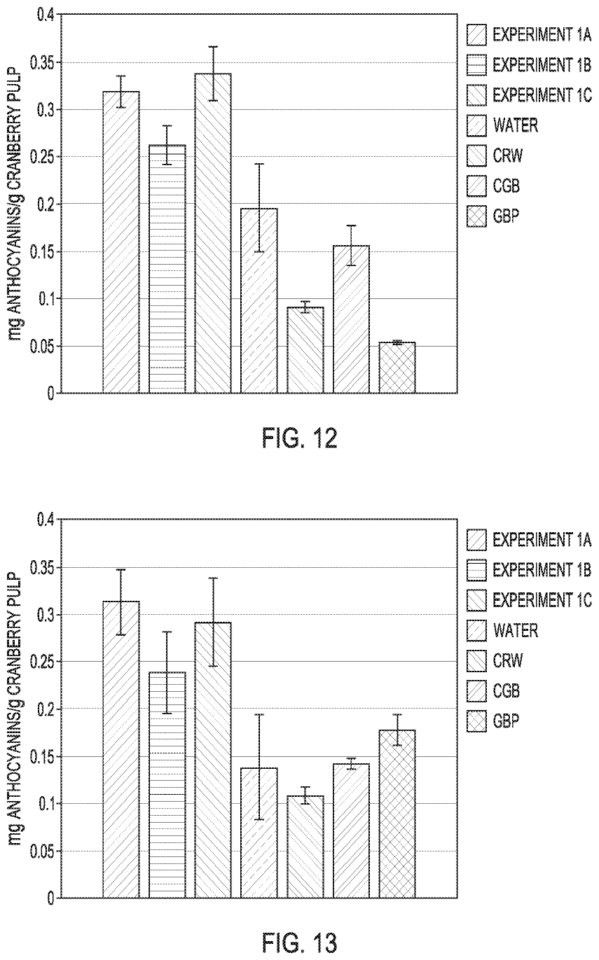

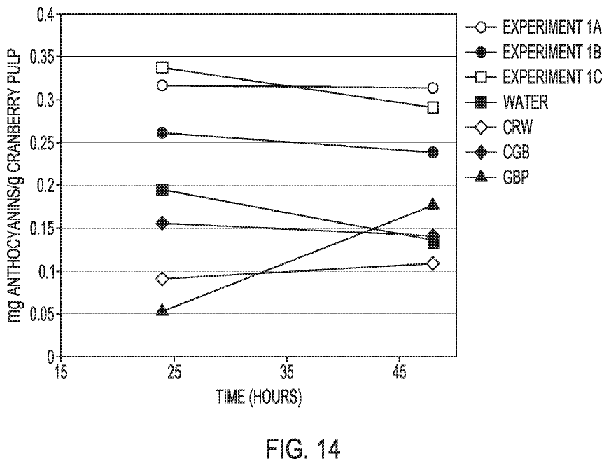

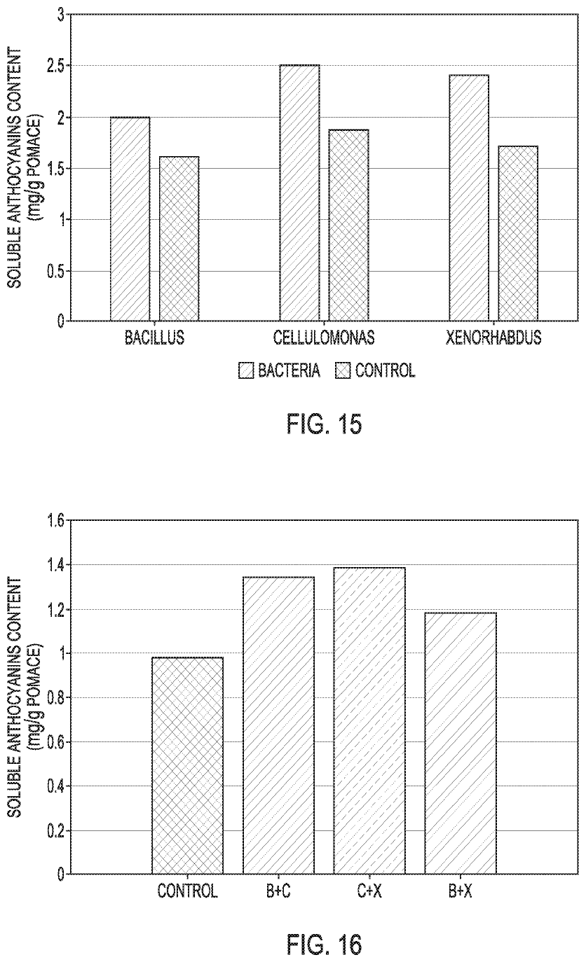

### Conclusions for FIG. 11:

1) All concentrations of bacteria improved aqueous extraction of anthocyanins from time-point 0 hr to 24 hr.
2) Condition 5 resulted in the highest concentration of extracted anthocyanins, but degraded nearly 15% between 24 and 48 hours.
3) Conditions 1-4 retained extracted anthocyanin content between 24 and 48 hours, but presented considerably less extracted anthocyanins than Experiment 1a.

### Conclusions

1) Experiment 1a resulted in the highest concentration of aqueous extracted anthocyanins with the least reduction in pigment from 24 to 48 hours.
2) Experiment 1c resulted in a high initial concentration of aqueous extracted anthocyanins, but significant degradation was observed from 24 to 48 hours.
Table 5: Final calculation of anthocyanin efficiency (% improvement over control).
Aqueous anthocyanin extraction improvement from time zero was compared for optimal bacterial concentration in Experiments 1a-c and a constant 8.3% (v/v) concentration for single bacterial species. Calculation was performed as described in methods. NA indicates anthocyanin loss below zero time point Legend: CRW, Corynebacterium glutamicum; CGB, Cellulomonas cellulans; GBP, Xenorhabdus nematophilia

TABLE 5

| | 24 hr | 48 hr |
|---|---|---|
| Experiment 1a | 435.2 | 10640 |
| Experiment 1b | 291.2 | 8960 |
| Experiment 1c | 377.6 | 9980 |
| CRW | NA | NA |
| CGB | 1327 | 4433 |
| GBP | NA | 226.4 |

### EXAMPLE 4

A significant percentage of antioxidant natural products are retained in the pulp, skin, and seeds of fruits and vegetables after processing. Microbial digestion of fruit pulp was examined as a mechanism for passively extracting high value natural products from agricultural waste products. Bacteria and fungi known to secrete enzymes capable of degrading the fruit waste matrix were incubated with fruit waste and the aqueous component was monitored for antioxidant content. The effect of microbial digestion on cranberry and cherry pulp resulted in improved aqueous extraction of the anthocyanins cyanidin and malvidin over controls. Specifically, enzymatic vs. microbial extraction were compared, the ratio of organisms, concentration, and duration of fermentation was determined, and total carbon and nitrogen content was determined.

To assess the efficacy of microbial digestion as a mechanism for freeing anthocyanins for aqueous extraction, thirteen microbial species were identified based on their secretion of enzymes capable of digesting the fruit waste matrix and were assessed both individually and in pairs. Extraction efficiency was compared to both aqueous controls and enzymatic digestion. It was found that the enzyme pullulanase improved extraction of anthocyanins 667% in 24 hours as compared to control. Single microbial treatments improved anthocyanin extraction up to 280% in 24 hours as compared to control. Anthocyanin concentrations overall were reduced from 24-48 hours. Single microbial treatments did not significant alter the carbon to nitrogen ratio of cranberry pulp with the exception of heavily sugared cranberry pulp. Heavily sugared (>50%) cranberry pulp saw a reduction in C/N ratio of 33%. However, this was due to an increase in nitrogen concentration rather than a decrease in carbon.

Dual microbial treatments improved anthocyanin extraction up to 435% in 24 hours and 10,640% in 48 hours as compared to control. Anthocyanin concentrations stabilized from 24-48 hours.

### EXAMPLE 5

The tested bacterial sources secrete one or multiple of five anthocyanin-effecting enzymes: cellulase, lipase, pullulanase, pectinase, and tannase (Table 6). Purified enzymes are purchased from Sigma-Aldrich and prepared to a standardized concentration in accordance with previously published methodology. Candidate bacteria with demonstrated enzyme secreting ability are compared against the enzyme activity using a standardized fruit waste (FW) generated by centrifugal juice extraction of whole fruit (e.g. cranberry, cherry, or grape). Samples are acquired over a time course experiment and soluble free anthocyanins quantified and characterized using established methodology. The free anthocyanins are quantified at the time of maximum extraction using the established spectrophotometric pH-differential method and comparison to authentic standards via High Performance Liquid Chromatography (HPLC) after acid hydrolysis of glycosides. Conservation of chemical character of the anthocyanins is confirmed via Liquid Chromatography-Mass Spectrometry (LC-MS). Chromatographic methods are performed in the SC Johnson Integrated Science Laboratory housed in the College of Science and Engineering at the University of Wisconsin-Parkside.

### Overview

1. Correlate microbial load (colony forming units, cfu) to enzyme activity (U/mg) against FW (fruit waste) substrate to obtain enzyme activity per microbial load metric (U/cfu) for further optimization.

2. Calculate cost differential between microbial source and equivalent purified enzyme on FW substrate.

3. Quantify free anthocyanins produced per cfu of bacteria prior to optimization of system (e.g. baseline determination).

After determination of baseline anthocyanin extraction and enzymatic activity per cfu, systems are evaluated for industrial scale goals:

1. Determine candidate bacteria with maximum anthocyanin extraction capacity (mg anthocyanins/cfu bacteria) from library of potential bacteria. Five bacteria from each enzyme secretion category (Cellulase, Lipase, Pullulanase, Pectinase, Tannase) are assayed against fruit waste (FW). At the completion of this experiment, candidate bacteria are reduced to a single candidate per enzyme category.

2. Assess potential for mixed microbial fermentation to further improve extraction efficiency.

Candidate bacteria are combined using a mixed microbial matrix (Table 7) to determine whether complimentary action increases, reduces, or has no change on the anthocyanin extraction efficiency. If multiple bacteria are found to increase anthocyanin extraction, an optimal bacterial load ratio experimental will be performed in order to determine the optimal ratio of bacteria to maximize anthocyanin extraction. At the completion of this experiment, a single candidate bacteria or single mixture of bacteria will move forward to complete optimization and scale up.

TABLE 6
Candidate microbes or families of microbes organized by enzymes
or metabolites produced: Use of designation sp. Indicates more
than one species within the genus produces enzyme of interest.
Microbial Candidates by Enzyme or Metabolite Production

| | | Pullulanase or | | |
|---|---|---|---|---|
| Cellulase | Lipase | Pectinase | Tannase | Organic Acids |
| Cellulomonas sp. | Candida sp. | Klebsiella sp. | Bacillus sp. | Lactobacillus sp. |
| Bacillus sp. | Pseudomonas sp. | S. cerevisieae | S. lugdunesis | Bidifidobacteria sp. |
| Thermobifida fusca | Xenorhabdus sp. | Streptomyces sp. | L. plantarium | Acetobacter sp. |
| Pseudomonas sp. | Y. lipolytica | Pseudomonas sp. | Acetobacter sp. | C. glutamicum |
| Streptomyces sp. | A. calcoaceticus | A. pullulans | S. cerevisieae | Brevibacterium sp. |

TABLE 7
Example Mixed Microbial Matrix. After reducing the
bacterial candidates to the best performing bacteria(s)
per candidate enzyme, bacteria will be combined with
bacteria secreting the remaining enzymes to determine
if multi-microbe systems offer increased efficacy for
the extraction of anthocyanins from fruit waste.
Example Mixed Microbial Matrix

| | Bacteria 1 | Bacteria 2 | Bacteria 3 | Bacteria 4 | Bacteria 5 |
|---|---|---|---|---|---|
| Bacteria 1 | | | | | |
| Bacteria 2 | X | | | | |
| Bacteria 3 | X | X | | | |
| Bacteria 4 | X | X | X | | |
| Bacteria 5 | X | X | X | X | |

### REFERENCES

Buchert et al., J. Science Food Agri., 85:2548 (2005).

Deller, Contribution of Agriculture to the Wisconsin Economy. AAE Staff Paper Series. University of Wisconsin-Madison (2014).

Dwyer et al., The Market Potential of Grape Waste Alternatives, 3: (2014).

Fernandes et al., J. Functional Foods, 7:54 (2014).

Foti, J. Pharm. Pharmacol., 59: 1673 (2007).

Ghafoor et al., J. Agri. Food Chem., 57:4988 (2009).

Gil-Chávez, et al., Comp. Rev. Food Sci. Food Safety, 12:5 (2013).

GNC. Life Extension Berry Complete. Available from: http://www.gnc.com/other-antioxidants.

He & Giusti, Annu. Rev. Food Sci. Technol., 1:163 (2010).

He et al., Food Chem., 204: 70 (2016).

Kähkönen & Heinonen, J. Agri. Food Chem., 51:628 (2003).

Kim & Lee, Extraction and Isolation of Polyphenolics, in Current Protocols in Food Analytical Chemistry, John Wiley & Sons, Inc. (2001).

Liazid et al., Food Chemistry, 124:1238 (2011).

Maier et al., Euro. Food Res. Tech., 227:267 (2008).

Panda et al., Crit. Rev. Biotechnol., 38:1 (2018).

Pandal, Nutraceuticals: Global Markets, BCC Research: Wellesley, Mass. p. 179 (2017).

Sullivan, F., Global Antioxidants Market, Forecast to 2021. 2017: https://ww2.frost.com/research/.

Tuck, B. a. W. G., Vineyards and Wineries in Wisconsin: A Status and Economic Contribution Report. 2014: University of Minnesota Extension.

White et al., J. Agri. Food Chem., 58:4030 (2010).

White et al., J. Agri. Food Chem., 59:4692 (2011).

Wijngaard et al., Food Res. Intl., 46:505 (2012).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to obtain water soluble anthocyanins, comprising:
a) providing a fruit or fruit seed, skin or pulp aqueous extract; and
b) contacting the extract and one or more microbes or one or more of isolated pullulanase, isolated cellulase, isolated lipase, isolated pectinase, or isolated tannase so as to yield a mixture comprising water soluble anthocyanins.

2. The method of claim 1 wherein the fruit is a cranberry or cherry.

3. The method of claim 1 wherein the fruit is blackberry, blueberry, grape, pomegranate, raspberry (red and black), tomato, or watermelon.

4. The method of claim 1 wherein the extract is contacted with pullulanase.

5. The method of claim 1 wherein at least one microbe is a bacterium or a yeast.

6. (canceled)

7. The method of any one of claim 1 wherein the extract is contacted with at least two microbes.

8. The method of claim 1 wherein the extract is contacted with one or more of microbes including Candida albicans, Saccharomyces cerevisiae, Staphylococcus lugdunesis, Klebsiella pneumoniae, Corynebacterium glutamicum, Lactobacillus plantarum, Cellulomonas cellulans, Xenorhabdus nematophilia, Pseudomonas aeruginosa, Bacillus subtilis, Bacillus cereus, Aureobasidium pullulans, or Brevibacillus laeterosporus or the extract is contacted with a combination of two or more microbes including Corynebacterium glutamicum, Lactobacillus plantarum, Cellulomonas cellulans, Xenorhabdus nematophilia, Pseudomonas aeruginosa, Bacillus subtilis, or Bacillus cereus.

9. (canceled)

10. The method of claim 1 wherein the extract is contacted with Corynebacterium glutamicum and Cellulomonas cellulans, Corynebacterium glutamicum and Xenorhabdus nematophilia or Cellulomonas cellulans and Xenorhabdus nematophilia.

11-12. (canceled)

13. The method of claim 1 wherein the extract is contacted with one or more microbes that secrete one or more of pullulanase, cellulase, lipase, pectinase, or tannase.

14. The method of any one claim 7 wherein the amount of each of the microbes is about 0.5% v/v to about 1.5% v/v, about 1.5% v/v to about 5% v/v or about 5% v/v to about 15% v/v.

15-16 (canceled)

17. The method of claim 1 wherein the amount of water soluble anthocyanins treated with pullulanase is increased relative to an extract treated with cellulase and/or pectinase.

18. A mixture obtained by the method of claim 1.

19. A method to isolate water soluble anthocyanins, comprising:
a) providing a fruit or fruit seed, skin or pulp aqueous extract;
b) contacting the extract and one or more microbes or one or more of isolated pullulanase, isolated cellulase, isolated lipase, isolated pectinase, or isolated tannase so as to yield a mixture comprising water soluble anthocyanins; and
c) isolating water soluble anthocyanins from the mixture.

20. The method of claim 19 wherein the fruit is a cranberry, cherry, blackberry, blueberry, grape, pomegranate, raspberry (red and black), tomato, or watermelon.

21. The method of claim 19 wherein the extract is contacted with pullulanase.

22. The method of claim 19 wherein the extract is contacted with one or more of microbes including Candida albicans, Saccharomyces cerevisiae, Staphylococcus lugdunesis, Klebsiella pneumoniae, Corynebacterium glutamicum, Lactobacillus plantarum, Cellulomonas cellulans, Xenorhabdus nematophilia, Pseudomonas aeruginosa, Bacillus subtilis, Bacillus cereus, Aureobasidium pullulans, or Brevibacillus laeterosporus or the extract is contacted with a combination of two or more microbes including Corynebacterium glutamicum, Lactobacillus plantarum, Cellulomonas cellulans, Xenorhabdus nematophilia, Pseudomonas aeruginosa, Bacillus subtilis, or Bacillus cereus.

23. (canceled)

24. The method of claim 22 wherein the extract is contacted with Corynebacterium glutamicum and Cellulomonas cellulans, Corynebacterium glutamicum and Xenorhabdus nematophilia or Cellulomonas cellulans and Xenorhabdus nematophilia.

25-26 (canceled)

27. The method of claim 19 wherein the extract is contacted with one or more microbes that secrete one or more of pullulanase, cellulose, lipase, pectinase, or tannase.

28. The method of claim 22 wherein the amount of each of the microbes is about 0.5% v/v to about 1.5% v/v, about 1.5% v/v to about 5% v/v or about 5% v/v to about 15% v/v.

29-30. (canceled)

31. The method of claim 21 wherein the amount of water soluble anthocyanins treated with pullulanase is increased relative to an extract contacted with cellulose and/or pectinase.
